(19) United States
(12) Patent Application Publication
(10) Pub. No.: US 2025/0302941 A1
(43) Pub. Date: Oct. 2, 2025

(54) Application of an Immunogenic or Vaccine Composition

(71) Applicant: EYE VACC, Angleur (BE)

(72) Inventors: Joël Blomet, Paris (FR); Norbert Schrage, Aix-la-Chapelle (DE); Frank Holzer, Saarbrucken (DE); Daniel Desmecht, Louveigne (BE)

(73) Assignee: EYE VACC, Angleur (BE)

(21) Appl. No.: 18/283,067

(22) PCT Filed: Mar. 22, 2022
(86) PCT No.: PCT/EP2022/057549

§ 371 Date: Sep. 20, 2023

(30) Foreign Application Priority Data
Mar. 22, 2021 (DE) 102021001467.7
Jun. 18, 2021 (EP) 21180477.8

(51) Int. Cl. A61K 39/215; A61K 9/00; A61K 39/00; A61P 31/14; C12N 7/00

(52) U.S. Cl. CPC A61K 39/215; A61K 9/0048; A61P 31/14; C12N 7/00; A61K 2039/541

(57) ABSTRACT

The present invention relates to an immunogenic or vaccine composition comprising a marker, for use thereof in prevention and/or treatment of an infection caused by at least one pathogen, characterized in that it is administered on the ocular mucosa and/or the urogenital mucosa.

In addition to the systemic immunity system, our bodies contain a mucosa associated immune system (called MALT, Mucosa Associated Lymphoid Tissue). The mucosa locally contain their own immune system, for example in the digestive tract (GALT, Gut Associated Lymphoid Tissue), in the nose (NALT, Nasal Associated Lymphoid Tissue), or even in the eye (CALT, Conjunctiva Associated Lymphoid Tissue).

In fact, the sites of entry of pathogens into the body are generally near the mucosa of the eyes, nose, mouth or gastrointestinal tract. Our bodies thus contain a large number of cellular and biochemical defense mechanisms directly at these mucosa, which activate on contact with pathogens. Typically the immune system of the mucosa comprises epithelial cells, innate immunity cells and dendritic cells which are the interface between the innate immunity and specific (acquired) immunity. The mucosa may therefore contain innate immunity cells, immunocompetent cells, memory cells and antibody-producing cells.

In the case of pathogens that colonize the mucosa and are highly contagious, the pathogen multiplies so rapidly after having colonized the mucosa that the infection of the mucosa and the deeper layers progresses faster than the organism can establish effective immunity for eliminating the pathogen. This is typically the case of the current infection by Severe Acute Respiratory Syndrome Corona Virus 2 (SARS-CoV-2). In fact, COVID-19 (or coronavirus disease 2019) mainly presents an infection and inflammation of the mucosa with a secondary severe general disease, as is also the case for the flu, herpes or poliomyelitis.

After having experienced a primary infection by a pathogen, the body develops a systemic (serum) immunity against this agent. Further, if the pathogen was in contact with the mucosa of the body, mucosal immunity is also developed, in addition to systemic immunity.

Vaccines which can develop mucosal immunity are already known. Those vaccines are administered essentially by nasal or oral route. They present the singular advantage of inducing a protective immune response, both in the mucosa and at the systemic level. They also target blocking crossing of the mucosa, gateway for most bacterial and viral pathogens. This is, for example, the case of the FluMist® flu vaccine which is administered by nasal spray, or even polio vaccines which are administered orally.

The flu virus (influenza A virus) vaccination, by ocular route, in ferrets was also studied in the article Eyedrop Vaccination Induced Systemic and Mucosal Immunity against Influenza Virus in Ferrets de Sangchul Yoon, Eun-Do Kim, Min-Suk Song, Soo Jung Han, Tae Kwann Park, Kyoung Sub Choi, Young-Ki Choi, and Kyoung Yul Seo; PLOS One. 2016; 11 (6): e0157634. Ocular route vaccination was also studied in mice by the team of Kyoung Yul Seo, Soo Jung Han, Hye-Ran Cha, Sang-Uk Seo, Joo-Hye Song, So-Hyang Chung, and Mi-Na Kweon (Eye Mucosa: An Efficient Vaccine Delivery Route for Inducing Protective Immunity; J. Immunol 2010; 185:3610-3619), even though mice are not natural hosts of the virus. However at this time no ocular vaccine has been approved.

In order to vaccinate the global population against various infections caused by at least one pathogen, in particular bacterial or viral infections, there is always a need for more and more effective vaccines. Advantageously, the vaccines must allow developing an immunity called sterilizing.

An effective vaccination is also understood to mean a vaccine administered in the recommended quantities, and/or administered correctly at the administration site. Vaccination errors have already been uncovered, in particular because of a bad injection site (for example subcutaneous administration of the BCG vaccine whereas it must be administered intradermally), or because of administration of vaccine doses greater than the recommended doses.

However, so far, there is no means by which to easily verify the quantity of vaccine administered and/or the correct application at the administration site.

There is therefore a need for effective vaccines, which can easily assure the good administration, in particular in terms of quantity administered and/or the administration site.

The present invention responds to this need with a new vaccination mode against infections caused by at least one pathogen which serves to (i) improve the immunity (double immunity-systemic and mucosal-providing in fact a better protection against the pathogen, both for the vaccinated host and for others because it could limit the risk of transmission of the pathogen), and (ii) assuring the good administration of the vaccine whether in terms of the quantity administered and/or the administration site.

The present invention thus provides a new vaccination mode against infections caused by at least one pathogen, more specifically bacterial and/or viral infections, that is more effective than other vaccination modes previously used, and which is simple to use.

The present invention serves to develop mucosal immunity against at least one pathogen, by targeting the ocular mucosa and/or the urogenital mucosa.

The present invention thus relates to an immunogenic or vaccine composition comprising a marker, for use thereof in prevention and/or treatment of an infection caused by at least one pathogen, characterized in that it is administered on the ocular mucosa and/or the urogenital mucosa.

The interest in administering such a composition is in establishing both an immunity in the mucosa and a serum immunity against at least one pathogen. This double immunity serves to better protect the organism against the pathogen and limits the risk of infections, without experiencing severe systemic secondary effects which may occur with some vaccines (for example the risk of thromboses observed with some COVID-19 vaccines administered intramuscularly). In fact, in the case of the present invention, since the serum immunity is acquired indirectly via immunization of the mucosa, a thrombotic accident is therefore not expected.

The interest of the presence of a marker in said composition serves also to detect and/or verify the quantity administered and/or the administration site, where this is done in order to assure a safe and effective application of the immunogenic or vaccine composition.

According to the invention, the expression “immunogenic composition” and/or “vaccine composition” is understood to mean a composition which induces an immune response against at least one pathogen after administration to the subject. The vaccine composition serves in particular to generate immunity, more specifically a protective and adaptive immune response against at least one pathogen, more specifically a virus and/or bacteria. This immune response may be humoral and/or cellular.

According to the invention, the term “a marker” is understood to mean any measurable or indicating substance which may be administered in an immunogenic or vaccine composition. It therefore involves a pharmaceutically acceptable marker. According to a specific embodiment, the marker serves to quantify and/or visualize the application of said immunogenic or vaccine composition on the ocular mucosa and/or the urogenital mucosa. More specifically, quantification is understood to mean the measurement of the quantity of immunogenic or vaccine composition administered. According to an embodiment, the marker is an indicating substance such as a colorant. As an example, fluorescein or indocyanine type colorants may be used.

According to the invention, the expression “an infection caused by at least one pathogen” is understood to mean a disease caused by one or more infectious agents.

According to the invention, the term “pathogen” is understood to mean any infectious agent capable of causing a disease in its host. More specifically, a pathogen is therefore understood to mean a virus, bacteria, parasite, fungus and/or a prion.

According to the invention, the expression “virus, bacteria, parasites, fungus and/or prion” is understood to mean a virus, bacteria, parasite, fungus and/or prion which are considered or could be considered as a pathogen for a body, preferably a human body. Therefore, this is typically not understood to mean bacteria beneficial to the body such as probiotics. More specifically, the expression “a virus, bacteria, parasite, fungus and/or a prion” is therefore understood to mean a pathogenic virus, bacteria, parasite, fungus and/or a prion.

The expression “infection caused by at least one pathogen” is understood to mean an infection caused by a pathogenic bacteria and/or virus and/or parasite and/or fungus and/or prion. According to a specific embodiment, the infection is a bacterial and/or viral infection, and more specifically the infection is either bacterial or viral. As an example, the infection is selected from: flu, herpes, infectious mononucleosis, poliomyelitis, salmonellosis, typhoid or paratyphoid fever, tuberculosis, cholera, Lyme disease, an infection caused by Staphylococcus aureus (in particular methicillin-resistant Staphylococcus aureus), an infection caused by pneumococcus, an infection caused by meningococcus, yellow fever, mumps, rotavirus gastroenteritis, measles, rubella, chickenpox, shingles, viral hepatitis, Japanese encephalitis, pertussis, diphtheria, tetanus, an infection caused by a coronavirus such as SARS or COVID-19, an infection caused by human papilloma virus, a prion disease such as Creutzfeldt-Jakob disease, infection caused by an amoeba such as amoebiasis, syphilis, fungal disease, malaria or an infection caused by GNMR (Gram-Negative Multidrug-Resistant) bacteria.

According to the invention, the expression “the prevention of an infection” is understood to mean prophylaxis. The administration of the immunogenic or vaccine composition according to the invention serves in particular to block or reduce the risk of developing said infection, and/or reduce as applicable the risk of developing forms of the disease referred to as severe (meaning with serious symptoms). The administration of the immunogenic or vaccine composition according to the invention could also block or reduce the risk of transmission of the pathogen, more specifically of the virus or bacteria, typically from one person to another. According to a specific embodiment, the immunogenic or vaccine composition for the prevention of infection according to the invention is administered to a subject who has not been contaminated, preferably not been infected, by the pathogen, preferably the virus and/or the bacteria.

According to the invention, the expression “the treatment of an infection” is understood to mean therapy. The administration of the immunogenic or vaccine composition according to the invention may in fact be considered in order to stimulate the natural defenses of the body, even though the person is a minima already contaminated by the pathogen, preferably the virus and/or the bacteria. According to a specific embodiment, the immunogenic or vaccine composition for the treatment of infection according to the invention is administered to a subject who is contaminated and/or infected, by the pathogen, preferably the virus and/or the bacteria.

Preferably, the invention thus relates to an immunogenic or vaccine composition comprising a marker, for use thereof in prevention of an infection caused by at least one pathogen, in particular a bacterial and/or viral infection, characterized in that it is administered on the ocular mucosa and/or the urogenital mucosa. According to an embodiment, the invention thus relates to an immunogenic or vaccine composition comprising a marker, for use thereof in treatment of a bacterial infection, characterized in that it is administered on the ocular mucosa and/or the urogenital mucosa. According to another embodiment, the invention thus relates to an immunogenic or vaccine composition comprising a marker, for use thereof in prevention of a viral infection, characterized in that it is administered on the ocular mucosa and/or the urogenital mucosa.

According to the invention, the expression “the ocular mucosa” is understood to mean the conjunctiva and the cornea. The conjunctiva is the transparent mucosal membrane which covers the inner surface of the upper and lower eyelids and which covers the anterior surface of the eyeball. More precisely, the ocular mucosa is understood therefore to mean both the tarsal conjunctiva and the bulbar conjunctiva, along with the conjunctival fornix.

According to the invention, the expression “urogenital mucosa” is understood to mean the mucosa of the urinary and/or genital system, both the male and the female system. More precisely, the urogenital mucosa is understood therefore to mean the urogenital tract.

The targeting of the ocular mucosa and/or the urogenital mucosa serves to target mucosal which (i) in themselves only participate weakly in the spread of the pathogen, but (ii) which at the same time have a very high immunocompetence, which serves to immunize the body very rapidly, in particular before an infection of the respiratory pathways. This embodiment further serves to reduce possible risks of serious side effects related to vaccination.

One of the inventors' nonlimiting hypothesis is in fact that infection of the eye by a pathogen leads to rapid formation of immunity, allowing the body to gain time such that when the contamination and/or the infection progresses in the body, in particular via the nose, the body is already immunized. This progression of the contamination and/or infection from the eye to the nose could be explained by the presence of the nasolacrimal canal which connects the nose to the eyes. An infection by the nasolacrimal canal has already been described in the case of keratitis caused by viruses (of keratitis epidemica type). The typical progression of this disease starts with an infection of the conjunctiva by droplets and is manifested by severe conjunctivitis followed by pharyngitis. This could also explain why subjects, in particular medical personnel, have developed systemic immunity against SARS-CoV-2 after having developed conjunctivitis positive to SARS-CoV-2.

Preferably, said immunogenic or vaccine composition is administered on the ocular mucosa. According to an embodiment, the invention thus relates to an immunogenic or vaccine composition comprising a marker, for use thereof in prevention and/or treatment of a bacterial infection, characterized in that it is administered on the ocular mucosa. According to another embodiment, the invention thus relates to an immunogenic or vaccine composition comprising a marker, for use thereof in prevention and/or treatment of a viral infection, characterized in that it is administered on the ocular mucosa.

According to another even more preferred embodiment, the invention thus relates to an immunogenic or vaccine composition comprising a marker, for use thereof in prevention of a bacterial infection, characterized in that it is administered on the ocular mucosa. According to another even more preferred embodiment, the invention thus relates to an immunogenic or vaccine composition comprising a marker, for use thereof in prevention of a viral infection, characterized in that it is administered on the ocular mucosa.

According to the invention, said immunogenic or vaccine composition may be administered in only one or in both eyes. Unlike intramuscular, subcutaneous or intradermal administration of the vaccine which requires an increased organization and medical effort (e.g. preparation of syringes, requiring availability of resuscitation equipment in case of severe reaction to the vaccination, etc.), the injection on ocular mucosa, typically by collyrium, is simpler and quicker to implement. In fact, the severe risks are reduced (for example risks of thrombosis as indicated above), as are the severe allergic risks which are principally seen as a tingling sensation in the eyes, tearing or even red eyes. Vaccination by collyrium further serves to vaccinate much more quickly and at a larger scale.

According to an embodiment, said immunogenic or vaccine composition comprises one or more substances serving to cause an immune response against one or more pathogens, in particular one or more viruses and/or one or more bacteria. Typically, said immunogenic or vaccine composition according to the invention comprises one or more antigens for said pathogen, in particular said virus or said bacteria. Preferably, said antigen(s) are specific to the pathogen responsible for the infection, in particular the virus or the bacteria responsible for the infection.

According to an embodiment, the immunity or vaccine composition serves to cause an immune response against at least one pathogen (in particular a virus and/or a bacteria), once it comprises at least one antigen for said pathogen, a microorganism (for example a virus or bacteria) which produces at least one antigen for said pathogen, or else it comprises the genetic material necessary for the expression of at least one antigen for said pathogen (typically an mRNA). In the first case, the antigen will be in direct contact with the mucosa following administration and in the second and third case a period of latency could be expected, the time after administration of the composition during which the antigen is produced. Preferably, said microorganism is not pathogenic per se, the only immune response that it may cause is that related to the antigen of said pathogen.

According to a specific embodiment, the substances/antigens serving to cause an immune response against said pathogen are selected from: (i) an inactivated virus, (ii) an inactivated bacteria or bacterial toxin, (iii) an attenuated virus, (iv) an attenuated bacteria or bacterial toxins, (v) an attenuated or inactivated parasite, (vi) an attenuated or inactivated fungus or fungal toxin, (vii) a prion, (viii) a genetically modified microorganism (expressing or able to express at least one antigen of a pathogen, optionally inactivated or attenuated), (ix) a messenger ribonucleic acid (mRNA) coding for one or more antigens of a virus or bacteria or parasite or fungus (such as a viral or bacterial protein), (x) deoxyribonucleic acid (DNA) coding for one or several antigens of a virus or bacteria or parasite or fungus (such as a viral or bacterial protein), (xi) a viral or bacterial or parasitic or fungal protein or one or more fragments thereof, or (xii) a fusion protein comprising a viral and/or bacterial and/or parasitic and/or fungal and/or prion protein, or one or more fragments thereof . . . . The substances/antigens serving to cause an immune response against said pathogen may also be (xiii) the pathogen per se, in particular a virus or bacteria that are not inactivated or not attenuated, (xiv) a recombinant cell (for example a dendritic cell) expressing or able to express one or more antigens of a pathogen, (xv) a DNA plasmid coding for one or more antigens of a pathogen (such as a viral protein), or (xvi) pseudo-viral particles comprising one or more antigens of a pathogen. A fragment of a viral and/or bacterial and/or parasitic and/or fungal and/or prion protein containing preferably the immunodominant epitopes or biosimilars thereof. According to an embodiment, the fragment of said protein is an immunogenic fragment. Said fragment and said protein may be recombinant.

According to an embodiment, a genetically modified microorganism is understood to mean a microorganism (preferably inactivated or attenuated) expressing or able to express one or more antigens of a virus or bacteria or parasite or fungus (such as a viral or bacterial protein), or a microorganism (in particular virus, bacteria or parasite) genetically modified to make them incapable of infecting a host body (for example by blocking them from penetrating a target cell or blocking multiplication thereof). It may for example involve a viral-vector type adenovirus.

According to a specific embodiment, the substances/antigens serving to cause an immune response against said virus and/or said bacteria are selected from: (i) an inactivated virus, (ii) an inactivated bacteria or bacterial toxin, (iii) an attenuated virus, (iv) an attenuated bacteria or bacterial toxin, (v) a genetically modified microorganism (preferably inactivated or attenuated) expressing or able to express one or more antigens of a virus or bacteria (such as a viral or bacterial protein), (vi) a messenger ribonucleic acid (mRNA) coding for one or more antigens of a virus or bacteria (such as a viral or bacterial protein), (vii) deoxyribonucleic acid (DNA) coding for one or several antigens of a virus or bacteria (such as a viral or bacterial protein), (viii) a viral or bacterial protein or one or more fragments thereof, or (ix) a fusion protein comprising a viral and/or bacterial protein, or one or more fragments thereof.

The composition according to the invention may comprise one or more substances (antigens) serving to provoke an immune response against one or more pathogens, in particular against one or more viruses, against one or more bacteria or against one or more viruses and one or more bacteria.

According to an embodiment, the various substances (antigens) may target different parts of the same virus or the same bacteria or the same fungus or the same parasite (for example different epitopes on viral or bacterial proteins), different strains of the same virus or the same bacteria or the same fungus, variants of the same virus or the same bacteria or the same fungus or the same parasite, or even several different viruses and/or several different bacteria and/or different fungi and/or different parasites (combined vaccine). The immunogenic or vaccine composition according to the invention may thus be monovalent or polyvalent. A monovalent immunogenic or vaccine composition protects against a single pathogen, whereas a polyvalent immunogenic or vaccine composition protects against several pathogens.

According to an embodiment, the expression “a viral or bacterial or fungal or parasitic protein” is understood to mean more specifically structural proteins and accessory proteins of the virus, or proteins involved in adherence, adhesion, invasion and/or internalization of the bacteria in a cell of the body, or of proteins involved in adherence, adhesion and/or invasion of the fungus in the tissue, or proteins and involved in adherence, adhesion, invasion and/or internalization of the parasite in the cell of the organism (such as a red blood cell), or even as applicable proteins secreted by the virus, bacteria, fungus and/or parasite. Preferably, said viral protein, bacterial protein, fungal protein or parasitic protein is selected from: an envelope protein, a matrix protein, a membrane protein or transmembrane protein, a surface protein or anatoxin. As an example, a bacterial or fungal surface protein may be an adhesin and a viral envelope protein may be a spike protein.

According to the invention, viral proteins is understood to mean native proteins of the virus (proteins of the virus such as found in nature), proteins that are mutated or variant compared to the native proteins or synthetic proteins (modified, mutated or not).

According to the invention, bacterial proteins is understood to mean native proteins of the bacteria (proteins of the bacteria such as found in nature), proteins that are mutated or variants compared to the native proteins or synthetic proteins (modified, mutated or not).

According to the invention, fungal proteins is understood to mean native proteins of the fungus (proteins of the fungus such as found in nature), proteins that are mutated or variants compared to the native proteins or synthetic proteins (modified, mutated or not).

According to the invention, parasite proteins is understood to mean native proteins of the parasite (proteins of the parasite such as found in nature), proteins that are mutated or variant compared to the native proteins or synthetic proteins (modified, mutated or not).

As an example, the pathogens responsible for the infections targeted by the present invention are the flu virus, the type 1 or type 2 herpes simplex virus, the Epstein-Barr virus, the poliovirus, bacteria of the Salmonella genus (preferably Salmonella enterica-typhi or paratyphi A, B, C), bacteria of the Mycobacterium genus (preferably Mycobacterium tuberculosis), Vibrio cholerae bacteria, bacteria belonging to the Staphylococcus genus (preferably Staphylococcus aureus), pneumococcus, meningococcus, yellow fever virus, mumps virus, rotavirus, measles virus, rubella virus, chickenpox or shingles virus, viruses referred to as A, B, C, D and E hepatitis, Japanese encephalitis virus, bacteria of the Bordetella genus (preferably Bordetella pertussis and Bordetella parapertussis), bacteria of the Corynebacterium genus (preferably C. diphtheriae, C. ulcerans, C. pseudotuberculosis), Clostridium tetani bacteria, a coronavirus such as SARS-CoV or SARS-CoV-2, or the human papilloma virus, a bacteria of the spirochete family (in particular Borrelia burgdorferi or Treponema pallidum).

The immunogenic or vaccine composition according to the invention is administered to a subject. According to an embodiment, said immunogenic or vaccine composition is used in the prevention and/or treatment of an infection caused by at least one pathogen in a human. According to the invention, a human may be a baby, child or adult.

According to another embodiment, veterinary applications of said immunogenic or vaccine composition according to the invention are conceivable. In such a case, the subject is then an animal.

According to an embodiment, said immunogenic or vaccine composition is administered to a subject in an immunologically effective quantity. Such a quantity may be determined by the practitioner. The expression “an immunologically effective quantity” is understood to mean a sufficient quantity for being effective at prevention and/or therapy, in particular in a subject needing such prevention or such treatment.

According to an implementation, said marker is present in an immunogenic or vaccine composition in a sufficient quantity for detection/visualization thereof.

According to an embodiment, said immunogenic or vaccine composition is administered to a subject who has not been contaminated, or even infected, by the pathogen, in particular the virus and/or the bacteria, responsible for the infection or else to a subject who has already been contaminated, even infected, by the pathogen, in particular the virus and/or the bacteria, responsible for the infection (that the person may have developed symptoms (mild or severe) or have been asymptomatic).

According to an embodiment, said immunogenic or vaccine composition is administered once or several times, preferably one, two or three times, on the ocular mucosa and/or the urogenital mucosa. According to a specific embodiment, said immunogenic or vaccine composition is administered at least twice on the ocular mucosa and/or the urogenital mucosa (in other words two doses of immunogenic or vaccine composition are administered). According to a specific embodiment, when the immunogenic or vaccine composition is administered several times, it is the same mucosa which is targeted: for example two or three administrations on the ocular mucosa, or two or three administrations on the urogenital mucosa. The length of time between the first and the other subsequent administration(s) is variable depending on the targeted pathogen, for example a few weeks for an immunogenic composition against the SARS-Cov-2 virus or a few months for an immunogenic composition against hepatitis A. According to an embodiment, said immunogenic or vaccine composition is administered on the ocular mucosa and/or the urogenital mucosa before or after at least one administration of an immunogenic or vaccine composition against the same pathogens via a different mode of administration (for example by intranasal or intradermal route). As an example, said immunogenic or vaccine composition is administered on the ocular mucosa after a first intramuscular administration (the intramuscular administration is here a first vaccination then the ocular administration is the first follow-up dose).

According to an embodiment, said immunogenic or vaccine composition is administered as a drop, lyophilizate or other dry form. According to an embodiment, said immunogenic or vaccine composition may also be administered as dry, powder, gel, nanoparticle or pomade composition form. Typically, when the administration is in drop form, that means that said immunogenic or vaccine composition is a liquid formulation, and when the administration is in lyophilizate or dried composition form, said immunogenic or vaccine composition is in powder form. Said powder may be applied directly to the mucosa, or else be applied on filter paper, polymers, gel, nanoparticles or mucosa or any other appropriate substances support which will be placed in contact with the ocular mucosa and/or urogenital mucosa. The immunogenic or vaccine composition will thus be transferred from the filter paper, polymer, gel or any other appropriate substance or support to the ocular mucosa and/or urogenital mucosa. Said dried composition may be obtained after freezing of the immunogenic or vaccine composition according to the invention and then dried with any appropriate technique (except for freeze-drying which serves to obtain a lyophilizate).

According to an embodiment, said immunogenic or vaccine composition is administered by using an applicator. An applicator serving in particular to make the administration of the composition easier, for example it makes it easier to instill drops (dropper type) or the powder, lyophilizate. The applicator may also be an ampoule, syringe, filter paper (with for example fluid or lyophilizate), a dropper bottle, a single dose applicator, a vaginal spiral or sponge type applicator, or even a fabric, a hygienic tampon comprising a modified outer surface (i.e. said surface comprising one or more substances serving to cause an immune reaction against SARS-CoV-2 such as a protein, a viral vector, etc.), or a condom comprising a modified external surface, etc.

According to an embodiment, the immunogenic or vaccine composition comprises or is made up of one or more substances (antigens) serving to cause an immune reaction against the pathogen, in particular a virus and/or a bacteria. According to an embodiment, it may be considered that the immunogenic composition according to the invention is included in a vaccine composition (a vaccine). The term “vaccine” or “vaccine against the pathogen responsible for the infection” may also be used in place of the expression “vaccine composition.” As an example, the immunogenic or vaccine composition according to the invention may be the Pfizer/BioNTech mRNA vaccine, generally called Bnt162b2 or Comirnaty® or the nonreplicating viral vector vaccine from AstraZeneca, generally called Vaxzevria®, AstraZeneca ChAdOx1-S or COVID-19 Vaccine when the virus is the SARS-CoV-2, the immunogenic or vaccine composition according to the invention may be the Dukoral® vaccine when the bacteria is Vibrio cholerae, or also the Revavix® vaccine (combination vaccine against the Corynebacterium diphtheriae bacteria responsible for diphtheria, against the Clostridium tetani bacteria responsible for tetanus and against a poliovirus responsible for poliomyelitis).

According to a preferred embodiment, sad vaccine composition comprises an adjuvant and/or an excipient.

According to an embodiment, said immunogenic or vaccine composition comprises a pharmaceutically acceptable vehicle.

The adjuvant and/or the excipient and/or the diluent and/or the pharmaceutically acceptable vehicle are those conventionally used. For example, the pharmaceutically acceptable vehicle may be a solvent or solution serving to dilute the composition before administration by ocular route or on the urogenital mucosa.

According to an embodiment, an immunogenic or vaccine composition according to the invention may further comprise one or more additional substances. Preferably, said additional substance is selected from:
a preservative,
a surfactant, and
an additive.

As an example, a preservative may be an antimicrobial preservative whose purpose is to block microbial contamination of the immunogenic or vaccine composition. According to the invention, the preservative is a preservative compatible with the mucosa.

In order to obtain an elevated antigenicity and therefore a greater immunological response, substances, for example surfactants, may be added to the immunogenic or vaccine composition in order to extend the exposure time on the ocular and/or urogenital mucosa, and possibly to allow calculating the exposure time of said composition on the mucosa.

Other substances, such as additives, may be added for conferring advantageous properties to the immunogenic or vaccine composition, for example a substance improving the penetration of the immunogenic or vaccine composition in the mucosa or a substance serving to extend the contact time between the mucosa and the composition (for example hyaluronic acid, one or more polymers for forming a hydrogel (e.g. carbomers), cellulose derivatives, etc.

The present invention also relates to a method for prevention and/or treatment of an infection caused by at least one pathogen in a subject comprising the administration of an immunogenic or vaccine composition on the ocular mucosa and/or the urogenital mucosa. More specifically, the present invention relates to a method for inducing a protective immune response against at least one pathogen, in particular a virus and/or a bacteria, comprising the administration of an immunogenic or vaccine composition on the ocular mucosa and/or the urogenital mucosa.

The present invention also relates to the use of an immunogenic or vaccine composition against at least one pathogen, in particular a virus and/or a bacteria, for the preparation of a medication intended to prevent and/or treat an infection caused by at least one pathogen, and where said medication is intended to be administered on the ocular mucosa and/or the urogenital mucosa.

### FIGURES

FIG. 1 shows the results for group 1 of hamsters.

FIG. 2 shows the results for group 2 of hamsters.

FIG. 3 shows the average of the weight of the hamsters in the four groups tested in Example 5.

### EXAMPLES

### Example 1: Examples of Liquid Compositions

Table 1 below summarizes the compositions according to the invention which may be used.

TABLE 1
Liquid compositions

| | | Viral | Number and type of |
|---|---|---|---|
| | Antigen or vaccine used | dose/concentration | applications |
| Composition 1 | The AstraZeneca nonreplicating | 0.1 × 10² to 10 × 10⁸ virus | One or more |
| | viral vector vaccine, generally | par mL | applications, preferably |
| | called Vaxzevria ® or | For example, doses of | three |
| | AstraZeneca COVID-19 vaccine, | 0.1 mL or 20 μL may be | Application on one or |
| | with addition of a coloring type | administered | both eyes |
| | marker | | |
| Composition 2 | The Gamaleya nonreplicating | 0.1 × 10² to 10 × 10⁸ virus | One or more |
| | viral vector vaccine, generally | par mL | applications, preferably |
| | called Spoutnik V or Gam- | For example, doses of | three |
| | COVID-Vac, with addition of a | 0.1 mL or 20 μL may be | Application on one or |
| | coloring type marker | administered | both eyes |
| Composition 3 | Viral proteins from SARS-CoV-2 | 1 × 10⁻¹² g/mL to 1 g/mL | One or more |
| | (S protein type), with addition of a | For example, doses of | applications, preferably |
| | coloring type marker. | 0.1 mL or 20 μL may be | three |
| | | administered | Application on one or |
| | | | both eyes |
| Composition 4 | Genetically modified non- | / | One or more |
| | pathogenic bacteria for producing | | applications, preferably |
| | a SARS-CoV-2 viral protein with | | three |
| | addition of a coloring type marker | | Application on one or |
| | | | both eyes |

### Example 2: Examples of Freeze-Dried Compositions

Table 2 below summarizes the compositions according to the invention which may be used.

TABLE 2
Freeze-dried compositions

| | | Number and type of |
|---|---|---|
| | Antigen or vaccine used | applications |
| Composition 5 | Freeze-dried viral proteins, | One or more applications, |
| | for example the S protein, | preferably three |
| | with addition of a coloring | Application on one or |
| | type marker. | both eyes |
| Composition 6 | Freeze-dried virus | One or more applications, |
| | (SARS-CoV-2, inactivated | preferably three |
| | or attenuated virus), with | Application on one or |
| | addition of a coloring | both eyes |
| | type marker | |

### Example 3: Immunization of Hamsters Against SARS-CoV-2 by Ocular Route

The hamsters have an ACE2 receptor. They may thus be contaminated by SARS-CoV-2 and become ill.

1. Comparison of Two Groups of Hamsters after Two Injections of Virus

Two groups of hamsters were studied.

In group 1, n=7 hamsters were contaminated with SARS-CoV-2 by nasal injection of a SARS-CoV-2 virus (B.1.214 strain) of 3×10⁶ and in group 2, n=7 hamsters were contaminated by means of a collyrium containing 3×10⁶ SARS-CoV-2 virus (B.1.214 strain). A coloring type marker may be added to the collyrium.

During the observation, the hamsters from group 1 became ill, recognizable by a significant weight loss, whereas the hamsters from group 2 did not become ill.

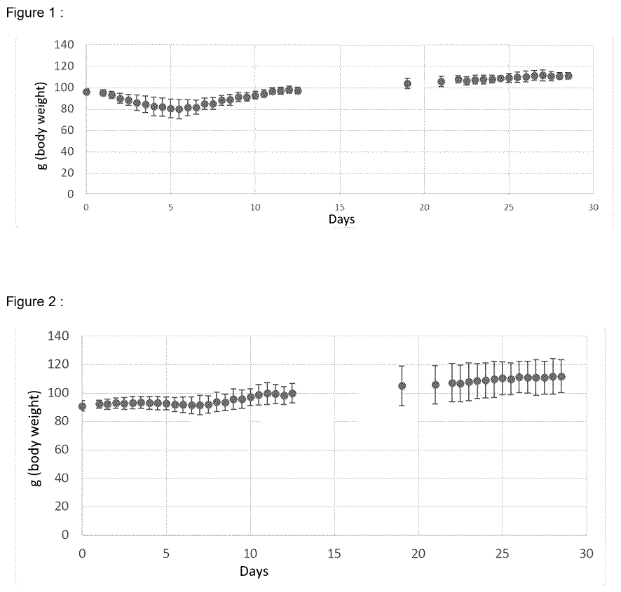

In fact, in FIG. 1 it can be seen that the hamsters from group 1 started to lose weight from day 2 with a maximum loss on day 5 and normalization on day 12; this indicates that the hamsters fell ill following SARS-CoV-2 contamination/infection by nasal injection. In contrast, in FIG. 2 it can be seen that the hamsters from group 2 do not lose weight after inoculation with SARS-CoV-2 by ocular route. This indicates that the animals remained in good health.

After 14 days, the two groups were again exposed to a nasal injection containing a SARS-CoV-2 viral dose of 3×10⁶.

In both groups, the hamsters continued to gain weight (see FIGS. 1 and 2). More specifically, at day 21, the animals from group 1 did not fall ill again after inhalation of the second SARS-CoV-2 viral dose. This means that they have become immune because of the illness developed during the first 10 days of the experiment.

Also, at day 21, the hamsters from group 2 did not fall ill after inhalation of the second SARS-CoV-2 viral dose. This means that the hamsters became immunized because of the first application of SARS-CoV-2 by ocular route.

The hamsters from group 2 thus acquired immunity with the epi-ocular application, which, unlike group 1, was acquired without developing a serious general illness.

2. Study of Hamsters after an Injection of Virus by Ocular Route

Further, the study of n=10 other hamsters which received a single application by ocular route of a composition containing a SARS-CoV-2 viral dose of 3×10⁶ (possibly in presence of a coloring type marker) showed that they developed a high production of neutralizing antibodies against the virus (at least 1640 in the serum). These results mean that by epi-ocular application of the virus, the hamsters did not develop disease and acquired an immunity against SARS-CoV-2 which is detectable in the serum.

### Example 4: Study of the Propagation of SARS-CoV-2 During Infection of the Nasal Cavity

The Inventors analyzed the propagation of the virus in hamsters who were contaminated with SARS-CoV-2 via a nasal injection, such as described in Example 3.

The Inventors thus observed that in the first 48 hours following entry of the virus in the nose, the infection progressed slowly with an infection of the nasal cavity only in the mucosa, with formation of aerosols. These aerosols can then infect the bronchus and the alveoli, in particular during inhalation, and contaminate other people during exhalation.

### Example 5: Vaccination by Means of Different Immunogenic or Vaccine Compositions

Four new groups of hamsters were studied.

In group 1, 20 μL of vaccine with Janssen/Johnson & Johnson nonreplicating viral vector (generally called Ad26COV2.S), taken directly from the vial, without any modification, were administered in the left eye of n=8 hamsters on D=1. After 14 days (D=14), 20 μL of the same vaccine, taken directly from the vial, without any modification, were again administered in the left eye of n=8 hamsters.

In group 2, 50 μL of vaccine with Janssen/Johnson & Johnson nonreplicating viral vector (generally called Ad26COV2.S), taken directly from the vial, without any modification, were administered intramuscularly in both hips of n=8 hamsters on D=1. After 14 days (D=14), 50 μL of the same vaccine, directly taken from the vial, without any modification, were again administered intramuscularly in both hips of n=8 hamsters.

In group 3, 20 μL of PBS (phosphate buffered saline) was administered in the left eye of n=8 hamsters on day D=1. After 14 days (D=14), 20 μL of PBS (phosphate buffered saline) was again administered in the left eye of n=8 hamsters.

In group 4, 20 μL of a solution containing 3×10⁶ of a SARS-CoV-2 virus (Wuhan-like variant of the SARS-CoV-2 virus), (an attenuated virus, not inactivated virus) were administered in both eyes of n=8 hamsters at 14 days (D=14) of the experiment.

After 14 additional days (D=28 since the beginning of the experiment), the four groups were contaminated intranasally with 200 μL of a solution containing 6×10⁶ TCID50 (Tissue Culture Infection Dose Fifty) of the SARS-CoV-2 virus (Wuhan-like variant of the SARS-CoV-2 virus).

A marker may be added to the vaccines and solutions administered in order to quantify and/or visualize their application.

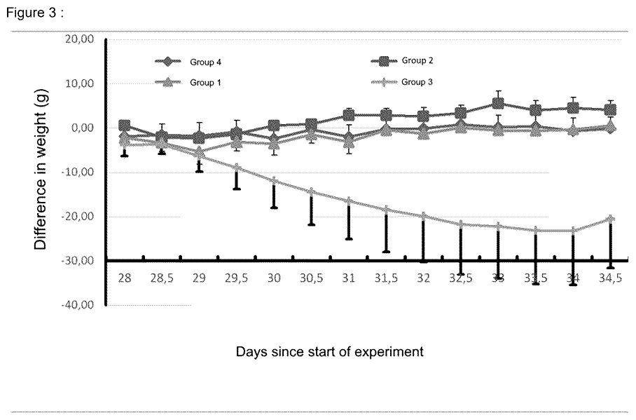

The weight loss of the animals (i.e. an indicator that the hamster was ill) was analyzed for each of the four groups. The results are presented in FIG. 3. The curves show the average weight of the hamsters in the four groups.

The results show the hamsters from groups 1, 2 and 4 are protected and do not fall ill, unlike the hamsters from group 3.

1. A method for the prevention and/or the treatment of an infection caused by at least one pathogen, wherein an immunogenic or vaccine composition comprising a marker is administered on the ocular mucosa and/or the urogenital mucosa.

2. The method according to claim 1, wherein said composition is administered on the ocular mucosa.

3. The method according to claim 1, wherein the marker serves to quantify and/or visualize the application of said immunogenic or vaccine composition on the ocular mucosa and/or the urogenital mucosa.

4. The method according to claim 1, wherein the marker is a colorant.

5. The method according to claim 1, wherein the infection is a bacterial and/or viral infection.

6. The method according to claim 1, wherein the composition comprises one or more substances serving to cause an immune response against one or more pathogens.

7. The method according to claim 1, wherein the pathogen is selected from: a virus, bacteria, parasite, fungus and/or prion.

8. The method according to claim 6, wherein the substance serving to cause the immune response is selected from: an inactivated virus, an inactivated bacteria or bacterial toxin, an attenuated virus, an attenuated bacteria or bacterial toxins, an attenuated or inactivated parasite, an attenuated or inactivated fungus or fungal toxin, a prion, a microorganism genetically modified from a bacteria, a parasite or a fungus, a messenger ribonucleic acid (mRNA) coding for one or more antigens of a virus or bacteria or parasite or fungus, deoxyribonucleic acid (DNA) coding for one or several antigens of a virus or bacteria or parasite or fungus, or a viral or bacterial or parasitic or fungal protein or one or more fragments thereof, a fusion protein comprising a viral and/or bacterial and/or parasitic and/or fungal and/or prion protein, or one or more fragments thereof, a pathogen, a recombinant cell expressing or able to express one or more antigens of a pathogen, a DNA plasmid coding for one or more antigens of a pathogen, or pseudo-viral particles comprising one or more antigens of a pathogen.

9. The method according to claim 8, wherein said viral, bacterial, parasitic, or fungal protein is selected from: an envelope protein, a matrix protein, a membrane protein or transmembrane protein, a surface protein or anatoxin.

10. The method according to claim 1, wherein said composition is administered in the form of a drop, lyophilizate, dried composition, powder, gel, nanoparticle or pomade.

11. The method according to claim 1, wherein said composition is administered by using an applicator.

12. The method according to claim 1, wherein said infection caused by at least one pathogen is selected from: flu, herpes, infectious mononucleosis, Lyme disease, poliomyelitis, salmonellosis, typhoid or paratyphoid fever, tuberculosis, cholera, an infection caused by Staphylococcus aureus, an infection caused by pneumococcus, an infection caused by meningococcus, yellow fever, mumps, rotavirus gastroenteritis, measles, rubella, chickenpox, shingles, hepatitis, Japanese encephalitis, pertussis, diphtheria, tetanus, an infection caused by a coronavirus, an infection caused by human papilloma virus, a prion disease, infection caused by an amoeba such, syphilis, fungal disease, malaria or an infection caused by GNMR bacteria.

13. The method according to claim 1, wherein said composition is administered in humans or animals.

14. The method according to claim 1, wherein said composition further comprises one or more additional substances.

15. The method according to claim 14, wherein the additional substance is selected from:
a preservative,
a surfactant, and
an additive.
